(19) United States
(12) Patent Application Publication
(10) Pub. No.: US 2004/0101868 A1
(43) Pub. Date: May 27, 2004

(54) Analysis method for hemochromatosis mutation

(76) Inventors: Veli Kairisto, Turku (FI); Gerard Donohoe, Dublin (IE); Jarkko Eskola, Turku (FI); Timo Korpela, Turku (FI)

Correspondence Address: BIRCH STEWART KOLASCH & BIRCH, PO BOX 747, FALLS CHURCH, VA, 22040-0747, US

(21) Appl. No.: 10/362,186

(22) PCT Filed: Aug. 21, 2001
(86) PCT No.: PCT/FI01/00733

(30) Foreign Application Priority Data
Aug. 21, 2000 (FI) 20001839

(51) Int. Cl. C12Q 1/68; C12P 19/34

(52) U.S. Cl. 435/6; 435/91.2

(57) ABSTRACT

The inventions deals with determination of the human hemochromatosis gene (HFE) mutation (C282Y of HFE protein), responsible for the disease of hereditary hemochromatosis. The invention is important in diagnosis and risk assessment for this disease. The method consists of a single-tube high-throughput PCR assay for the detection of C282Y. We invented that it is advantageously possible to combine three concepts each known separately in prior art from different sources: allele specific PCR, mutagenically separated PCR, and amplicon identification by specific dissociation curves. Analysis can be performed in either a conventional or fluorescence-detecting thermocycler using the same primers, reactant constituents and cycling protocol. PCR products are identified either by their length or melting temperature (Tₘ). Primer cross reactions are prevented by deliberate primer: primer and primer: template mismatches. This homogenous assay is fast, reliable, robust, automatable and does not require fluorescent oligonucleotide probes. It is therefore significantly more economic and straightforward approach for HFE genetic screening than used in the prior art.

### FIELD OF INVENTION

The invention relates to a genetic test for identifying subjects carrying one or more of copies of mutated gene causing hereditary hemochromatosis. More specifically, the invention concerns novel design of oligonucleotide probes to be used with DNA amplifying methods that can be exploited to analyze the presence or absence of the mutated gene with an improved reliability, economy, and convenience.

### BACKGROUND OF INVENTION

Genetic hemochromatosis (GH) is the most common genetic illness in the northern hemisphere with a prevalence of approximately 5 per 1000 [Felitti V J. and Beutler E. Am J Med Sci 1999;318:257-68]. Most GH patients are homozygous for a one base difference at cDNA position 845 of the hemochromatosis gene (HFE). This single nucleotide polymorphism (SNPs) is a G to A transition that results in a tyrosine for cysteine substitution at amino acid 282 (C282Y) of the HFE protein [Feder J N, at al. Nat Genet 1996; 13:399-408]. SNPs are defined as single nucleotide substitutions and small unique base insertions and deletions [Gu Z, et al. Hum Mutat 1998;12:221-5]. These stable mutations represent the most common form of DNA sequence variation, and they occur at a rate of 0.5-10 per every 1000 base pairs within the human genome. SNPs can serve as genetic markers and some can also significantly contribute to the genetic risk for common diseases [Schafer A J. and Hawkins J R. Nat Biotechnol 1998; 16:33-9].

Even today testing for certain SNPs can provide critical diagnostic information for management of patients and their families. Established examples of this include for example, the APOE alleles in atherosclerosis [de Knijff P. and Havekes L M. Curr Opin Lipidol 1996;7:59-63] and Alzheimer disease [Roses A D. Curr Opin Neurol 1996;9:265-70], the F5 1691G→A allele in deep-venous thrombosis [Bertina R M,et al. Nature 1994;369:64-7], the CCR5Δ32 in resistance to HIV infection [Dean M, et al. Science 1996;273:1856-62], the BRCA mutations in breast and ovarian cancer [Casey G. Curr Opin Oncol 1997;9:88-93] and the 845G→A allele in GH [Feder J N, at al. Nat Genet 1996; 13:399-408].

Methods of diagnosis, markers and primers for a single-pair polymorfism causing hemochromatosis were disclosed in patent U.S. Pat. No. 5,712,098 [Zenta et al.]. In said invention, however, relatively short conventional primers flanking the single base-pair mutation, were employed. This method has several drawbacks including the need for separate assays for determining whether the mutation is homozygous or heterozygous. In addition, the primers used cause in certain cases false results.

The ability to carry out rapid DNA analysis, in order to determine the mutational status or genotype of an individual, has thus become an increasingly important task for the clinical diagnostic laboratory. Consequently, there is a need for a fast, cheap, accurate, reliable, robust, high-throughput and easy to set up assay, for the identification of clinically significant SNPs. Currently a wide variety of different methods are available for detecting single base changes in a DNA molecule. These techniques include; restriction isotyping [Stott M K, et al. Clin Chem 1999;45:426-8], single-strand conformation polymorphism [Bosserhoff A K, et al. Biotechniques 1999;26:1106-10], oligonucleotide ligation assay [Feder J N, at al. Nat Genet 1996; 13:399-408], heteroduplex analysis [Jackson H A, et al. Br J Haematol 1997;98;856-9] and allele-specific oligonucleotide hybridization probes [Beutler E, at al. Blood Cells Mol Dis 1996; 22:187-94]. However, a simple and cost efficient way to determine the genetic status of an individual is by the use of allele specific PCR. In this method an oligonucleotide primer is specially designed to match one allele but mismatch the other allele at or near the 3′ end. If the DNA polymerase cannot extend a primer with a 3′ mismatch this means that one allele is preferentially amplified over the other. The specificity of the allele specific primers can be further enhanced by engineering a deliberate base change very close to their 3′ end. In order to identify a bi-allelic polymorphism two physically separate PCR reactions are required for each analysis. In addition, a pair of control primers that amplifies an independent fragment is usually included in the reaction to ensure that the PCR reaction itself was successful. This method is known by a variety of names, allele specific amplification (ASA), amplification refractory mutation system (ARMS) and PCR amplification of specific alleles (PASA). Based on this principle, a number of methods have been developed to detect the C282Y mutation in the HFE gene.

An enhanced approach know as PCR amplification of multiple specific alleles (PAMSA) or mutagenically separated PCR (MS-PCR) [Rust S, et al. Nucleic Acids Res 1993;21:3623-9], allows both allele specific oligonucleotides to be coamplified and differentiated using only a single PCR reaction. Cross reactions between the different allele specific primers are avoided by the use of deliberate mismatches at or near the 3′ and 5′ end of the primers. In comparison to ASA the need for an internal control primer set is eliminated and the cost and labor of the techniques is reduced by about one half. Merryweather-Clarke et al. [Merryweather-Clarke A T. et. al. Br J Haematol 1997;99:460-3.] have recently applied this method to the detection of the HFE C282Y genotype. However this technique as well the aforementioned methods are still not ideally suited to large-scale analysis because they require a laborious post PCR processing step.

The problems of low throughput and the requirement for postamplification manipulations have been overcome by the recent development of a new type of PCR machine that can monitor the PCR reaction in real time. These machines are composed of a thermal cycler coupled to a fluorescent detector and are capable of PCR amplification with simultaneous amplicon analysis [Ririe K M. Et. al. Anal Biochem 1997;245:154-60.]. Currently the most favored approach for the detection of the specific PCR products has been the use of sequence specific dual labeled fluorescent probes (TaqMan probes), in combination with the 5′-3′exonuclease activity of Taq polymerase. An alternative approach that does not require the 5′-3′exonuclease acitivity of the polymerase, is the use of a two-probe system [Mangasser-Stephan K, et.al. Clin Chem 1999;45:1875-8.]. However for both these strategies there is a rather high cost involved in the purification and labeling of the probes. In addition, the design of the probes can be problematic especially if the target region is AT rich. And finally, optimization can be a complicated and difficult as there is a need to have more than two oligonucleotides in the PCR reaction.

A simple and cost-effective method for concurrent DNA amplification and detection, is to use a fluorescence double stranded DNA specific binding dye, such as SYBR Green I, in combination with allele specific primers. Products are detected by their characteristic melting profiles. A product melting profile is generated after the PCR reaction by monitoring the fluorescence of the SYBR Green I dye as the temperature passes through the amplicons denaturation temperature. Melting profiles are dependent upon the GC content, length and sequence of the PCR products.

### BRIEF DESCRIPTION OF THE DRAWINGS

FIG. 1. Schematic diagram of the allele specific PCR primers used to detect the C282Y HFE gene mutation.

Both the HFEW and HFEW2 primers differ from the HFEM primer by five nucleotide bases. In the figure above the first difference, which occurs at the 3′ nucleotide, is illustrated by a grey box with black lettering, whereas the other four differences between the primers are represented by a black box with white lettering. These mismatches ensure that misprimming and cross reactions between primers and template are prevented.

FIG. 2. Comparison of C282Y genotyping by (A) GeneAmp 9600 and (B) MJ research PTC-200 DNA Engine.

PCR products from both thermocyclers were analyzed by a short dissociation protocol using the GeneAmp 5700 Sequence Detection system. Fluorescence melting curves were converted to derivative melting peaks by plotting the negative derivative of the fluorescence with respect to temperature against temperature [−(df/dT) vs T]. The derivative melting peaks are shown for a HH sample (peak 845 G), a wildtype sample (peak 845 A), a heterozygote sample (peaks 845 G and 845 A), and a no-template control. The 845 A peak has a higher temperature value than the 845 G peak due to a greater GC content.

FIG. 3 C282Y HFE genotyping by allele specific PCR gel-based electrophoresis using three different thermocyclers

The PCR mixture contains the allele specific primers HFEM (80 bp) and HFEW2 (113 bp). Each PCR reaction was performed using the same reactants and cycling protocol. Lane 1 50-bp ladder. Lanes 2,6 and 9, PCR was performed by the MJ research PTC-200 DNA Engine; Lanes 3,7 and 10, PCR was performed using the Perkin-Elmer/Cetus 480 DNA thermocycler. Lanes 4,8 and 10, PCR was performed with the PE-Biosystems GeneAmp 5700 Sequence Detection system.

FIG. 4. Sample to sample and within sample variation of the C282Y derivative melting peaks.

A total of 68 PCR reactions comprising ten duplicate wild-types, ten duplicate heterozygotes, ten duplicate non template controls and two quadruplicate mutant homozygotes samples were analyzed using the GeneAmp 5700 Sequence Detection system. All 68 dissociation curves for each individual genotype are superimposed in the figure above.

FIG. 5 Scatter graph of the different replicate C282Y HFE genotypes and the non-template controls (NTC).

The scatter graph was generated by plotting the area under the dissociation curve between temperatures 82° C.-84° C. (Peak 1) on the x-axis. Similarly, the area under the dissociation curve between temperatures 85° C.-87° C. (Peak 2) was plotted on the y-axis. Using fixed cut-off limits for area under peak 1 (vertical line crossing x-axis at 2.0) and peak 2 (horizontal line crossing y-axis at 1.5) the three different genotypes and NTCs can be automatically scored.

### DETAILED DESCRIPTION OF INVENTION

The present invention describes a new method to detect single mutation with PCR reaction. The essence of the invention is the design of specific mismatch primers enabling to detect both normal and mutated alleles in one PCR reaction. The reaction mixture does not necessarily need to be subjected to any further analysis. The developed method is more reliable and more economical than described in the prior art. The assay can be carried out without opening the reaction vessel since the amplification products can be analyzed through the transparent or opalescent tubes. This is a very remarkable advantage because PCR diagnostic laboratories tend to be contaminated readily by the reaction products. However, the PCR products can be also subjected to traditional detection such as electrophoresis on agarose gel. The design of the described oligonucleotide primers are novel and are based on totally new principle. The oligonucleotides are exceptionally long (25-70 bp) with several missmatches but, however, the preferred primers do not extend to the polymorfic nucleotide of the intron as in the previous invention described in the U.S. Pat. No. 5,712,098 (Tsushihashi Zenta et al.).

The basic embodiment of the invention involves the finding that it is possible to combine of all the assay principles of [Newton C R, et al. Nucleic Acids Res 1989;17:2503-16.], [Rust S, et al. Nucleic Acids Res 1993;21:3623-9.27] and [Germer S,and Higuchi R. Genome Res 1999;9:72-8.] whenever the oligonucleotide primers of the assay are carefully designed. The applicability of the new assay concept is demonstrated within the detection of the C282Y HFE polymorphism. The same principles can be exploited in detection of practically any other point mutation.

HFE polymorfism is a good example of a disease that can greatly benefit from a simple and cheap DNA screening test to identify carriers and affected individuals. The disease is characterized by a life-long excessive accumulation of iron and has a high morbidity and mortality rate resulting from damage to cardiac, hepatic and endocrine tissues. This disease however, is preventable if identified and treated early by simple phlebotomy, which removes excess iron [Felitti V J. and Beutler E. Am J Med Sci 1999;318:257-68.]. The recent finding that C282Y heterozygosity may be associated with an increased risk of cardiovascular death adds further to its public health importance. Relatively wide occurrence and possibility to avoid totally the harms of the disease, make screening of hemochromatosis among population sensible and thus economic aspects are especially pronounced.

Most individuals with hemochromatosis (>80%) are homozygous for the missense mutation C282Y. In contrast, compound heterozygosity or homozygosity for the H63D mutation, is associated with hemochromatosis only with very low penetrance. Not more than approximately 1% of the compound heterozygotes will develop hemochromatosis. This figure is even less for H63D homozygotes. Thus approximately 99% of compound heterozygotes that would be found, in either relatives of C282Y homozygotes, or in the general population, would be false positives [Felitti V J. and Beutler E. Am J Med Sci 1999;318:257-68.]. Therefore we believe that H63D genotyping, is only relevant for C282Y heterozygotes, and only in those cases were the clinical suspicion of hemochromatosis remains, as assessed by biochemical tests such as, ferritin and transferring saturation. Thus, for a clinical laboratory engaged in hemochromatosis screening, the first-line genetic test should be C282Y genotyping.

Currently the most widely used method for C282Y detection is PCR—restriction isotyping. However, the use of a four-hour digestion step post PCR, the manual gel loading of samples and the possibility of misinterpretation due to partial digestion, makes this method time consuming, labor intensive and prone to error. Recent results have shown that this assay, when used with the primers of [Feder J N, at al. Nat Genet 1996; 13:399408.], has the potential to wrongly classify a C282Y heterozygote as a C282Y homozygote. This is caused by a newly identified single nucleotide polymorphisms (5569 G/A) located in the binding region of the [Feder J N, at al. Nat Genet 1996; 13:399408.] antisense primer.

These problems as described above are avoided by the new method described in the present invention. Our gel-based AS-PCR assay accurately determines wild type, heterozygous, as well as homozygous HFE samples. In order to eliminate the need for post-PCR processing, we tested the new assay with SYBR Green I and the GeneAmp 5700 Sequence Detection System. However due to the overlap in melting profiles for both the wildtype and mutant products, identification of the different HFE C282Y genotypes proved impossible. This occurred even though there was a difference in length of 20 bp between the HFEM and HFEW PCR products. Subsequent analysis of the products revealed a GC content of 59% for the HFEM amplicon and 60% for the HFEW amplicon.

The Tm of a PCR product is mostly dependent on its GC content and DNA length. The HFE amplicons did not differ significantly in length. Therefore the failure to differentiate their melting peaks was probably due to their very similar GC content. To overcome this, we increased the GC content of the wildtype product to 65%. This yielded a single wild type melting peak of approximately 87° C. and since the mutant product has a 4° C. lower melting peak, product discrimination was easily facilitated.

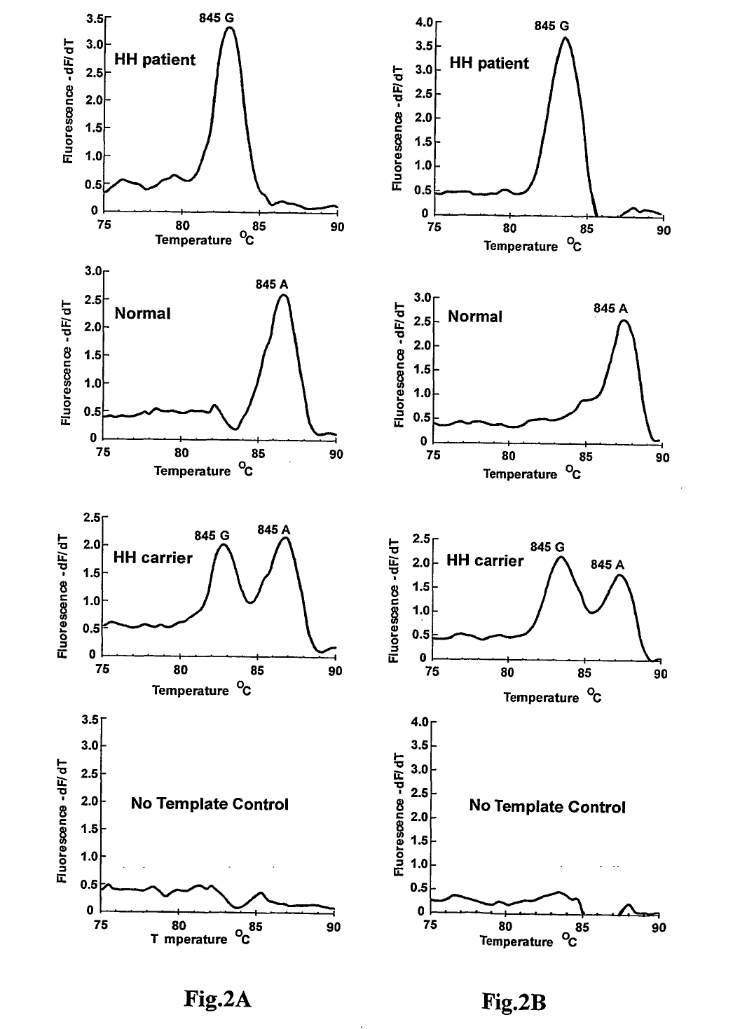

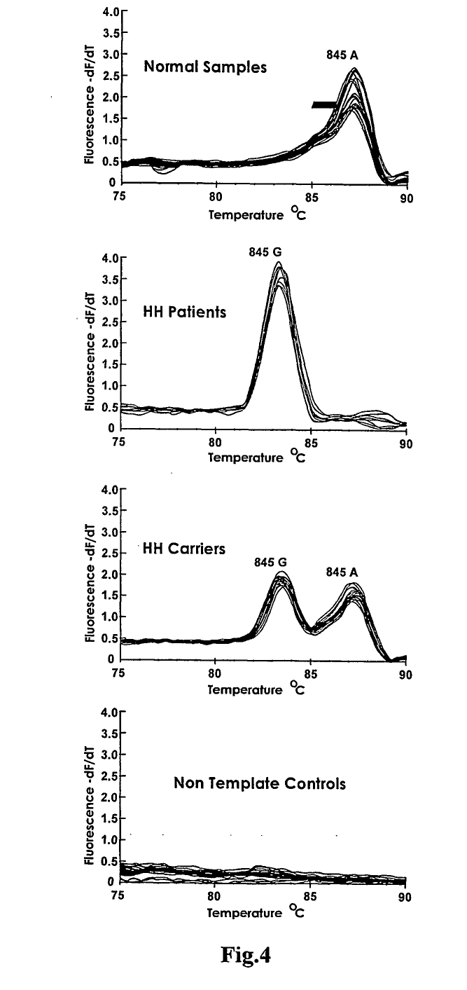

Thus we have shown that a one-step, one-tube HFE genotyping assay can be performed using SYBR Green I, allele specific oligonucleotides, and a fluorescence-detecting thermocycler (FIG. 2A.). Furthermore, the reliability and robustness of this new technique was demonstrated by the high degree of reproducibility for each allele melting curve (FIG. 4). In contrast to other multiplex formats involving fluorescent oligonucleotide probes, the reagent costs for this assay are minimal. Moreover, in comparison to a standard PCR reaction, the only additional reagent requirement, is the inexpensive SYBR Green I DNA binding dye.

The capital costs of the GeneAmp 5700 Sequence Detection system are high. Therefore, to maximize productivity, the machine should be used for a variety of different assays. Consequently, machine time available to individual users will become scarce. We have shown, however, that it is possible to perform the PCR step using a standard thermocycler. And that a subsequent 20 minute melting curve analyze of the products by the GeneAmp system, gives unequivocal results (FIG. 2B.). Thus an added benefit of this genotyping technique is its versatility, which yields substantial saving in machine time.

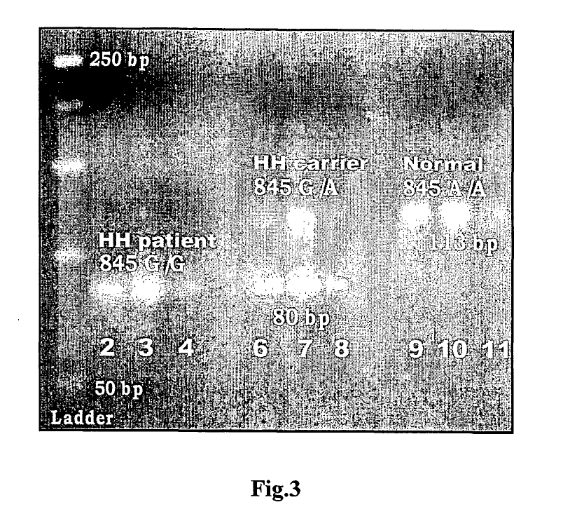

Still few clinical laboratories have access to a real-time thermocycler. Therefore, we have designed this assay so that the same primers, reactants and cycling protocol can be used for either the GeneAmp SDS system or a conventional thermocycler. Thus even in the gel-based format (FIG. 3), this HFE assay can be setup with minimal investment and one person can genotype a large number samples in one working day.

One potential drawback of the current method, which may be a reason that other researchers have overlooked the present assay concept, is that SYBR GREEN I, binds to all amplification products including primer dimers and this could cause difficulty in identifying the intended amplicon. Primer dimer formation did occur, but fortunately after optimization of the reaction conditions, the wrong products were differentiated from the desired amplicons by differences in melting temperatures. Moreover the primer dimer products were eliminated through a combination of increased annealing temperature, alteration in the ratio of primers and a reduction in the number of PCR cycles. We found that it was necessary to use the longer AS-primer at a lower concentration than the shorter one. This probably reflects the greater annealing advantages of longer primers at high temperatures.

The experimental results show that the assay format of the present invention represents a very straightforward and economical way to genotype C282Y HFE locus. The fact that some SNP targets are very GC rich, could make the optimization and discrimination of their PCR products difficult. Thus an obvious aid for designing good PCR primers for use in this system, is a method that can accurately predict the melting temperature of the resulting PCR product. We calculated the theoretical Tm for the HFE products. Comparison of the theoretical Tm with the measured Tm, revealed an overestimation of 2.4° C. for the HFEM amplicon and 3.7° C. for the HFEW2 amplicon. This discrepancy probably results from the addition of SYBR GREEN I, which has been shown to affect melting curves in a concentration dependent fashion.

In conclusion a new homogenous single tube HFE genotyping method, that combines the principles of AS-PCR, MS-PCR and amplicon identification by SYBR GREEN I melting curves, has been developed. We have shown that the new method is accurate, reliable, versatile, and easy to implement. Thus, our assay represents a cost effective approach to HFE genotype determination and thus represent a significant methodological and economic improvement over the prior art.

Next the invention will be further illustrated by specific examples focusing onto detection of hemocrohromatosis.

Sequenc Listing

| | |
|---|---|
| nnatccaggcctgggtgctccacctnny: | SEQ ID NO:1 |

### EXAMPLE 1

We tested >200 subjects for the C282Y mutation. This group comprised subjects taken randomly from the general Finnish population using blood samples routinely collected for the examination of the blood count. The blood samples were anonymized, retaining only birth year and sex. The study protocol was in concordance with the Helsinki Declaration of 1975, as revised in 1983. Genomic DNA was extracted from 3 ml of EDTA-anticoagulated blood.

DNA Extraction

The DNA extraction was carried out using the salting-out principle

Blood cells were lysed with 7.5 ml of Tris buffer 1 (Trizma Base 1.58 g, KCl 746 mg, MgCl2×6H2O 2.95 g, EDTA 744 mg, Triton X-100 25 ml and deionized water added to a final volume of 1000 ml, pH 8.0, adjusted with 1.0 mol/L HCl). After centrifugation (7 min, 2400 g) the pellet was washed with Tris buffer 1 and centifugated (7 min, 1200 g). Next, the pellet was lysed with 660 μL of Tris buffer 2 (Trizma Base 158 mg, KCl 74.6 mg, MgCl2×6H2O 95.2 mg, EDTA 74.7 mg, NaCl 2.3 g, sodium dodecyl sulfate 1 g, deionized water added to a final volume of 100 ml, pH 8.0, adjusted with 0.1 mol/L HCl) and incubated for 15 min at 56° C. Cellular proteins were removed by precipitation, after addition of 300 μL of 5 mol/L NaCl (centrifugation 7 min, 560 g). DNA was isolated by ethanol precipitation and incubated for one hour at 4° C. in Tris-EDTA buffer (Trizma Base 158 mg, EDTA Titriplex 3 37.2 mg, deionized water added to a final volume of 100 ml, pH 8.0, adjusted with 0.1 mol/L HCl). The DNA concentration was then measured by spectrophotometry at 260 nm, and samples were diluted to a final concentration of 20 mg/L.

### EXAMPLE 2

Prefererred primers contained two distinct forward primers: a wild type primer HFEW (5′-GGGGGGCCCCGGGCCCAGATCACAATGAGGGGCACATCCAGGCCTGGGTGCTCCACCTCGC-3′), and a mutant primer HFEM (5′-TGATCCAGGCCTGGGTGCTCCACCTGCT-3′). The method uses also a reverse primer that amplifies both alleles, a common primer HFECOM (5′-CAGGGCTGGATAACCTTGGCTGTACC-3′), and a fluorescent dye SYBR Green I, that can detect double-stranded DNA (dsDNA).

Each PCR reaction mixture contained the following reagents in a final volume of 25 μL: 50 ng of genomic DNA, PCR reaction buffer (10 mmol/L Tris-HCl, pH 8.8, 1.5 mmol/L MgCl2, 50 mmol/L KCl, and 1 mL/L Triton X-100), 5 mM dNTP, 1 U of DyNAzyme II DNA Polymerase (Finnzymes), 5 pmol of both common and wild type primers, 20 pmol of mutant primer and 2.5 μL of SybrGreen I 1:10000 (Molecular Probes). Negative control reactions containing water in place of DNA were included in each batch of PCR reactions to exclude appearance of contamination. To investigate the versatility of the method, the PCR amplification was carried out in three different thermocyclers (MJ research PTC 200, Perkin Elmer480, and Perkin Elmer GeneAmp 5700).

The PCR amplification profile was as follows: initial denaturation at 95° C. for 4 min, 32 cycles with denaturation at 96° C. for 30 s, combined annealing and extension at 71° C. for 30 s.

### EXAMPLE 3

### Product Analysis

The analysis of PCR products on gel was done as follows. Amplified product (10 μL) was mixed with 2.5 μL of 6× gel loading dye type I (Sigma) and separated in a 2.75 % agarose gel (Sigma) that contained 0.1 mg/L ethidium bromide (Bio-Rad). The samples were electrophoresed for 50 min at 12 V/cm in a minigel (Hoefer), using 0.5× Tris-borate-EDTA running buffer (1× Tris-borate-EDTA: 90 mmol/L Tris borate, 2 mmol/L EDTA, pH 8.0, and 0.08 mg/L ethidium bromide). The amplicons were sized using a 50-bp molecular mass marker (Roche). In GeneAmp 5700 the analysis of the real-time fluorescence signal from SybrGreen I unspecifically bound to double-stranded DNA was performed by GeneAmp 5700 software (Perkin Elmer). The derivative of the dissociation curve data was used to separate the two PCR products.

### EXAMPLE 4

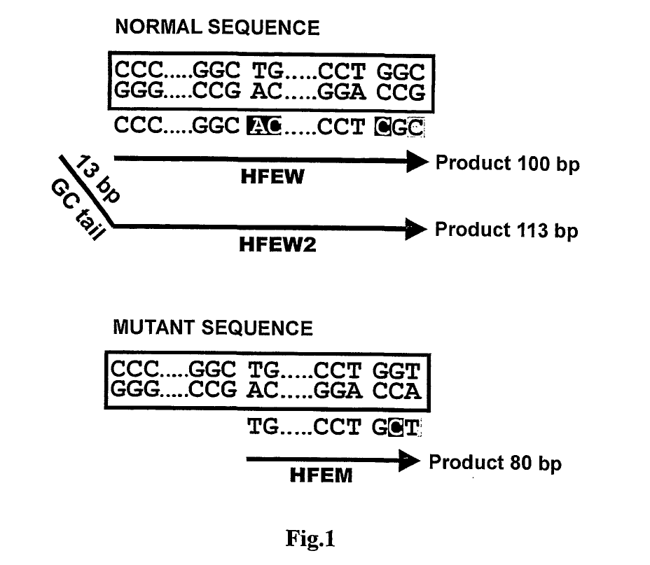

A schematic representation of the different oligonucleotide primers used for genotyping the C282Y locus is shown in FIG. 1. Initially three oligonucleotide primers were designed based on the NCBI Genbank HFE CDNA sequence (accession number U91328). In order to allow product identification from the single reaction mix, the allele specific primers were designed. The two forward allele specific primers (HFEW, HFEM) were 48 and 28 bp long, respectively, and the complementary primer (HFECOM) was 26 bp in length. Mispriming and cross reactions were prevented by the introduction of deliberate mismatches between primers and template.

The first nucleotide difference (C or T) between the allele specific primers HFEW and HFEM is preferably located at their 3′ terminal base. To ensure the specificity of these primers, a DNA polymerase that lacks the 3′ exonuclease proof reading activity (DyNAzyme II) was used in the PCR reaction. The second primer base change, (G to C) generates a purine/pyrimidine primer/template mismatch, and this prevents amplification of the non-matching allele specific primer. This mismatch is located three bases from the 3′ end of HFEW2 and two bases from the 3′ end of the HFEM primer. Two additional nucleotide changes (A and C) were made to the HFEW primer. The changes are located at the same position as the last two 5′ nucleotides of the HFEM primer. They prevent the generation of possible spurious products, which could otherwise occur by the annealing and extension of the HFEM primer to the first round product of HFEW.

We tested the primers using a single PCR reaction in a standard thermocycler Perkin-Elmer/Cetus 480. The primers were found to be highly specific for the C282Y mutation as wild-type, mutant homozygotes, and heterozygotes samples were readily distinguishable. Analysis by slab gel electrophoresis revealed that the wild type samples generated the expected 100 bp with the HFEW primer and no product was amplified with the HFEM primer. Similarly the HFEW primer generated no product with the mutant homozygote sample but as expected the HFEM primer generated a band of 80 bp. And for the heterozygote sample both the 80 and 100 products were amplified due to the presence of one copy of the mutant and wild-type alleles.

### EXAMPLE 5

Next we tested the possibility of using designed primers with the PE-Biosystems GeneAmp 5700 Sequence Detection system. This machine can simultaneously amplify and detect DNA targets using the simple principle of SYBR Green I fluorescence and melting curve analysis. As the HFEW primer has a higher GC content than the HFEM primer, we initially tested the possibility of differentiating these allele specific primers by melting curve analysis. However this did not prove possible because the melting profiles for both primer products were not distinguishable from each other due to the overlap in the rate of change of their fluorescence values (data not shown). In order to overcome this we added a thirteen base pair GC tail to the HFEW primer (HFEW2). We intentionally added only a small GC tail so as not to substantially alter the composition of an already well functioning primer.

FIG. 2a shows the results for the HFEW2, HFEM and HFECOM primers with the GeneAmp 5700 Sequence Detection system. For each C282Y sample the allele specific primers accurately distinguished between mutant homozygote, wildtype and heterozygote. The melting of the sample homozygous for the 845 G showed a mark change (decrease) in fluorescence between 85° C. and 87° C., with a clear maximum rate of change at 86° C. In contrast, the sample homozygous for the 845 A allele, showed a mark decrease in fluorescence between 82° C. and 84° C., with a clear maximum rate of change at 83° C. The heterozygous sample contained both fluorescent melting peaks due to the presence of amplicons derived from both alleles. Analysis of the products by standard slab electrophoresis revealed that the GC-tailed primer HFEW2 was specific for the wild-type G allele whereas the short primer HFEM was specific for the A allele.

### EXAMPLE 6

Next we tested the possibility of using a standard thermocycler (MJ research PTC-200 DNA Engine) to amplify the C282Y locus. We then used a short twenty minutes dissociation protocol on the 5700 machine to analyze the PTC-200 products, the results of which are presented in FIG. 2b. These results clearly show that the melting peaks produced by the products of either thermocycler are practically identical.

### EXAMPLE 7

The versatility of the gel-based assay was assessed by running the PCR in three different thermocyclers; PE Biosystems GeneAmp PCR System 9600, Perkin-Elmer/Cetus 480 DNA thermocycler and the MJ research PTC-200 DNA Engine. Each thermocycler analysis was performed with exactly the same samples, reactant concentrations and cycling conditions. The gel based results are depicted in FIG. 3. These results demonstrate that the assay functions in different thermocyclers without the need for any modifications.

### EXAMPLE 8

To test the reproducibility of the fluorescence melting curves, we analyzed ten wild-type, and ten heterozygotes samples in duplicate, as well as two mutant homozygotes samples in quadruplicate (FIG. 4). Each allele melting curve was found to be highly reproducible, as the sample to sample and with in sample variation of the melting curves were <0.5° C. The robustness of the technique was evaluated by analyzing >200 samples and all samples tested gave an unambiguous C282Y HFE genotype. The validity of the method was confirmed by an outside laboratory, which analyzed samples comprising all three C282Y HFE genotypes.

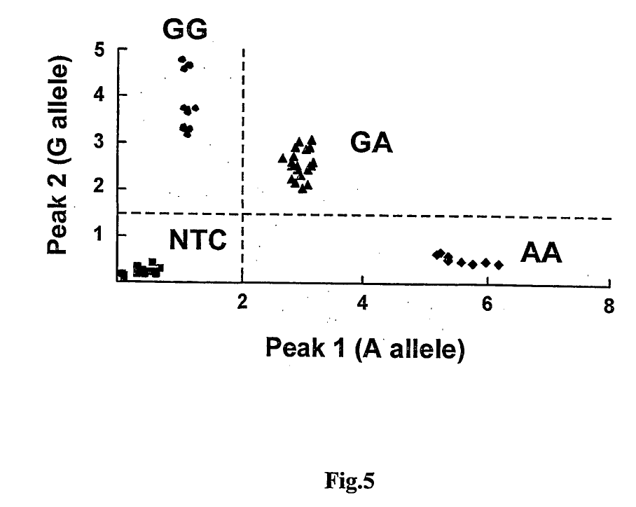

The SDS 5700 software allows the export of numeric dissociation curve data to other software. We exported the data into Microsoft Excel and designed a program macro which calculated the area under the dissociation curve. Subsequently, a scatter graph was generated where the area under the dissociation curve between temperatures 82° C.-84° C. was plotted on the x-axis. And the area under the dissociation curve between temperatures 85° C.-87° C. was plotted on the y-axis. This generated a graph in which the three C282Y genotypes and the non-template controls separated into four discrete clusters with definable limits (FIG. 5). Using this customized Excel sheet it was possible to automate the process of genotype scoring.

1. A method of diagnosis of human hereditary hemochromatosis gene (HFE) mutation of an individual, the method being characterized by:
a DNA sample from an individual is investigated for the presence or absence of a mutation in the hfe gene at position 845,
application of covalently non-labeled oligonucleotide primers flanking to HFE gene position 845, one of them amplifying healthy genomic dna and another amplifying mutated genomic DNA,
by adjusting the binding strength of the modified oligonucleotide primers by optimizing the nucleotide sequence near the 3′-end of the primers by deliberate base mismatches, adding a 5-50 bases long cg-tail to the 5′-end of the primers; or adjusting the composition or concentration of ions in the solution of the polymerase chain reaction, in a way that the gene products, after subjecting genomic DNA to gene amplification process, deviate as regards to their melting temperatures and/or degrees of amplification, effecting that the amplicons can be analysed:

2. A diagnostic method according to claim 1, characterized by:
specifically designed oligonucleotide primers, hybridizing with genomic DNA samples to be analysed for human hereditary hemochromatosis (HFE) gene mutation, which primers contain in their sequences nucleotides not pairing with the subject DNA,
the melting temperatures and amplification degrees, or alternatively amplicon lengths and amplification degrees, of each possible gene products are adjusted by a proper design of non-pairing nucleotides within the primers.

3. Nucleotide primers according to claim 2, wherein the oligonucleotide primers are characterized by:
the 3′-end of an oligonucleotide lies in the close vicinity of the position 845 of human hereditary hemochromatosis (HFE) gene,
use of one forward primer including the sequence (SEQ ID NO: 1) for amplification of normal DNA and one forward primer including the sequence (SEQ ID NO: 1) for amplification of mutant DNA with one reverse primer amplifying both alleles, or alternatively,
the use of said primers except that of one forward primer additionally contains in its 5′-end additional pairing or non-pairing oligonucleotide tail
any of the oligonucleotides do not hybridize with the intron sequences of the HFE gene

4. A diagnostic method according to claim 1, characterized by:
by utilizing of the oligonucleotides described in claims 2 and 3, the human hereditary hemochromatosis (HFE) gene under study is amplified by techniques known in prior art while the amplification products are detected by electrophoretic method or by methods able to differentiate the melting temperatures of the gene products,
after a single amplification reaction, the gene products can be analyzed and the results show whether the gene material is healthy, homo-, or heterozygous as to the single nucleotide polymorphism of the HFE gene.

5. A diagnostic method according to claims 1-4, characterized by:
Oligonucleotide primers, defined in claim 3, are exploited with a DNA to be diagnosed in a single-tube reaction with a fluorescent dye, preferably SYBR Green I, or related dye able to monitor the presence or absence of double-stranded DNA,
the reaction mixture is subjected to a DNA-amplification procedure while the fluorescence of the monitoring dye is followed continuously or analyzed after completion of the amplification reaction,
the judgement of possible abnormalities in human hereditary hemochromatosis (HFE) gene is based on the existence of the position and number of peaks in the obtained DNA melting curve,
derivative fluorescence melting curves of healthy individuals, as to hereditary hemochromatosis, show one peak as well as the patients of the disease under consideration, but the peaks appear at distinct melting temperatures,
carriers of hereditary hemochromatosis show two peaks of derivative fluorescence melting curves, one peak corresponding to normal and the other one to patient of hereditary hemochromatosis,
the analysis reaction is performed in a single vessel and the result of the analysis shows whether the studied DNA belongs to a healthy, homo- or heterozygotic individual of hereditary hemochromatosis.

6. A diagnostic method according to claim 1-5, characterized by:
Oligonucleotide primers, defined in claim 3, are exploited with a DNA sample to be diagnosed,
the said reaction mixture with due amplification reagents are subjected to a DNA amplification process in a single, preferably sealed reaction vessel,
after the amplification process, the reaction products are analyzed with by electrophoretic techniques known in prior art, revealing the sizes of specific amplicons, followed by their analysis based on their location compared with standard DNA markers, the bands on the electrophoresis support show whether the sample belongs to a healthy, homo- or heterozygotic individual in respect to hereditary genetic hemochromatosis.

7. A diagnostic method according to claim 1, which employes at least one primer including sequence depicted in SEQ ID NO:1:
nnatccaggcctgggtgctccacctnny,
or a longer sequence obtained by addition other nucleotide sequences at its 5′-end or a single nucleotide at its 3′-end.
